(19) United States
(12) Patent Application Publication
(10) Pub. No.: US 2007/0270472 A1
(43) Pub. Date: Nov. 22, 2007

(54) Amino, Amino Acid or Peptide Conjugates of Retinoic Acid

(76) Inventors: Raphael Beumer, Loerrach (DE); Jochen Klock, Freiburg (DE); Juergen H. Vollhardt, Ramlinsburg (CH); Philippe Emmanuel Maillan, Eschentzwiller (FR); Stefan Stoeckli, Basel (CH)

Correspondence Address: NIXON & VANDERHYE, PC, 901 NORTH GLEBE ROAD, 11TH FLOOR, ARLINGTON, VA, 22203, US

(21) Appl. No.: 11/631,322

(22) PCT Filed: Jul. 1, 2005
(86) PCT No.: PCT/EP05/07134

§ 371 Date: Jan. 25, 2007

(30) Foreign Application Priority Data
Jul. 9, 2004 (EP) 04016239.8

(51) Int. Cl. A61K 31/20; A61K 31/40; A61K 31/4172; A61K 31/44; A61Q 19/00; C07C 233/00; C07D 207/02; C07D 213/60; C07D 233/66

(52) U.S. Cl. 514/351; 514/399; 514/423; 514/558; 546/300; 548/338.1; 548/533; 554/37

(57) ABSTRACT

The invention provides the use of retinoyl derivatives for the cosmetic treatment or prophylaxis of wrinkles, skin aging and/or for thickening the epidermis.

Amino, amino acid or peptide conjugates of retinoic acid The present invention is directed to amino, amino acid or peptide conjugates with Retinoic acid and to compositions containing them. The compositions are preferably topical preparations, more preferable pharmaceutical or cosmetic formulations, in particular cosmetic formulations. It was found that the peptide, amino or amino acid conjugates of Retinoic acid are able to prevent or treat age or stress related signs of skin aging.

Human skin undergoes certain normal cornification processes which give the skin its characteristic appearance. Casual factors or external factors such as a raw climate, wind, photo-damage and irritation triggered by the sun, rain and snow, however, disturb this normal condition of the skin, and there appears a roughness, a formation of scales (for example on the scalp), an excessive keratinization and similar phenomena. Furthermore, in the course of aging of the skin various signs appear that are especially reflected by a change in the structure and function of the skin. One of these signs is the appearance of fine lines and deep wrinkles, the size and number of that increase with age. The micro relief of the skin becomes less uniform and is of anisotropic nature. In parallel with age the skin becomes more sensitive towards disturbing influences, either intrinsic or extrinsic, which may result in itching, redness or even darker spots, particular on hands and the facial area due to pigmentation disorders. These unwanted signs may lead to an undesired age judgment of a person.

Cosmetic preparations are essentially useful for skin care. One aim of skin care in the cosmetic sense is to strengthen or rebuild the skin's natural function as a barrier against environmental influences (e.g. UV-light, dirt, chemicals, microorganisms) and against the loss of endogenous substances (e.g. water, natural lipids, electrolytes). If this function becomes impaired, increased resorption of toxic or allergenic substances or attack by microorganisms may result, leading to toxic or allergic skin reactions.

Another aim of skin care is to compensate the loss of lipids and water by the skin caused by daily washing. This is particularly important, if the natural regeneration ability is inadequate.

Furthermore, skin care products should protect against environmental influences, in particular against sun and wind, and delay skin aging.

Strengthening or thickening of the epidermis together with an optimized skin barrier lipid synthesis can rebuild the skin's barrier ability and is therefore of significant cosmetic value. Reduced transepidermal water loss (TEWL) is a sign of an intact lipid barrier, which acts also as first defense line to protect against the appearance of skin wrinkles.

Another strategy to fight wrinkles is to stimulate the collagen synthesis in the dermis. A number of degenerative processes act on the collagen matrix and is triggered by extrinsic factors like UV radiation, pollution in general and particular cigarette smoke or intrinsic factors leading to any chronic or sub chronic inflammation. Destruction and/or impaired repair efficacy leads to a denser and less elastic macro structure of the dermis, which in turn leads to the formation of deep wrinkles. Enhancing the de novo synthesis of collagen or other structural proteins of the dermis is considered a valuable therapy to reduce the existing wrinkles and to protect against the appearance of new wrinkles.

Of particular importance for anti-aging cosmetics is to inhibit the senescence of skin cells in order to keep their regular metabolic level on a constant and beneficial level.

DE 2102586 discloses Retinamides for topical pharmaceutical uses to treat cancer, precancerous lesions, acne, psoriasis and other changes involving increased keratinization of the skin as well as eczema. Cosmetic applications, particular to prevent and treat age related effects are not disclosed. The document is also not disclosing any peptide or amino acid derivatives of Retinoic acid.

U.S. Pat. No. 4,108,880 and U.S. Pat. No. 4,190,594 disclose amino derivatives of Retinoic acid for the use as sun filters. The derivatives have been animal tested and proven to be metabolic inactive compared to Retinoic acid. Amino acid or peptide conjugates are not mentioned.

WO 99/50240 discloses polyethoxylated retinamides for cosmetic preparations to treat wrinkles and freckles due to skin disorders like cancer and acne. The compounds are tested to have a good skin penetration. Non-oxygen containing amines, particular non-ethoxy-amines as well as amino acid and peptide conjugates are not mentioned.

DE 4032187 discloses cysteine conjugates of Retinoic acid for treatment of diseases of mucous membranes. Cosmetic applications are not disclosed.

Shealy et. al. published [Journal of Medicinal Chemistry (1988), 31(1), 190-6] the chemo preventive activity in vitro of some amino acid conjugates of Retinoic acid on murine leukemia and human epidermoid carcinoma cells. Healthy skin cells have not been treated.

WO 2004010966 and JP 2001039997 provide cosmetic formulations to improve wrinkles and to activate cells with stable and water soluble glucose amine derivatives of Retinoic acid. The document does not disclose any amino acid conjugates or alkyl amino derivatives.

EP 1297830 discloses various alpha- or beta-amino acid derivatives for prevention and treatment of tissue damage by ozone; however the document is silent in terms of amides with Retinoic acid.

WO 00/15188 reports about specific peptides for the healing, hydrating and improving skin appearance as well as treatment of skin aging. For enhancing the lipophilicity the N-terminal amine is supposed to carry a fatty acid chain with 2 to 22 carbons.

EP 0864563 discloses the use of N-acyl-hydroxyamino acid esters for protection of skin and hair by designing biomimetic compounds of ceramides. Ceramides contribute significantly to the skin lipid barrier and require two long chain fatty acids one of them having preferentially more than 16 carbon atoms. The document does not disclose or claim amides of Retinoic acid.

EP 1159952 suggests cosmetics containing Hydroxyproline or acylated Hydroxyproline. The document does not disclose conjugates with Retinoic acid.

U.S. Pat. No. 5,492,894 discloses compositions with tripeptides up to hexapeptides to treat skin wrinkles. The peptides can optionally be modified by various substitution patterns at N- as well as the C-terminus. Modification with Retinoic acid is not disclosed

Retinol and derivatives thereof in combination with a certain skin lightening acid is known to be useful in the repair of photo-damaged skin or the prevention of photo-damage to skin following the exposure to UV-light, see e.g. WO 94/09756.

While a variety of technologies exist to prevent and to fight the signs of skin aging and to improve the appearance of the skin, there is still a demand for more efficacious ingredients.

The problem to be solved by the present invention is the provision of compounds, of compositions containing these compounds, in particular of cosmetic preparations which are particularly useful for treating and/or preventing wrinkles and thickening of the epidermis and regulating sebum production, but also preparations which are useful against other conditions which are observed with skin aging due to environmental or other external influences or due to age. The compounds should have an excellent activity.

This problem is solved on the basis of the unexpected finding that alkyl amides and amino acid or peptide conjugates of Retinoic acid have excellent activity for treating and preventing wrinkles and thickening the epidermis, but also for ameliorating the effects of aging of the skin, which may be caused by external or environmental hazards or by the natural aging of the skin. Some of these compounds have never been used before for cosmetic purposes, and therefore the use of these compounds for achieving a cosmetic effect is novel. The most preferred compounds of the present invention are novel per se.

Accordingly, the present invention provides the use of a compound represented by general formula (I) 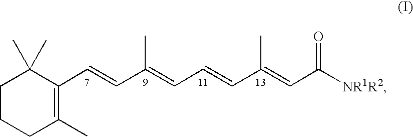
wherein

R¹ and R² independently of each other represent hydrogen, or a C₁-C₃₀-hydrocarbon group or a residue 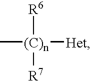
or R¹ and R² form together with the nitrogen atom to which they are attached a 5- to 8-membered saturated or unsaturated ring which contains besides the nitrogen atom carbon atoms and optionally 1 or 2 further heteroatoms selected from nitrogen, oxygen and sulfur atoms and which is unsubstituted or substituted with 1 to 3 substituents, independently selected from C₁-C₆ alkyl groups, OR⁸ groups, or C₁-C₆ alkoxy groups, each of the above alkyl and alkoxy groups being optionally substituted by 1 to 3 groups OR⁸, or

NR¹R² represents a residue 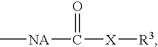
wherein 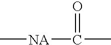
represents the residue of an amino acid or of a peptide which is bonded to the moiety 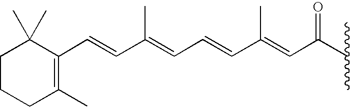
(the retinoyl moiety) over the N-terminus of the amino acid or the peptide and the peptide is composed of 2 to 6, that means 2, 3, 4, 5 or 6 amino acids,

—X— is —O— or —NR⁵—

R³ is hydrogen, a C₁-C₁₆ hydrocarbon residue or a residue PAG-R⁴,

PAG is a residue of a polyalkylene glycol,

n is an integer of O to 3,

Het is a 5 to 8-membered saturated or unsaturated heterocycle which contains 1 to 3 heteroatoms, independently selected from nitrogen, oxygen and sulfur and which is optionally substituted with 1 to 4 substituents, independently selected from C₁-C₆ alkyl groups, OR⁸ groups, or C₁-C₆ alkoxy groups, each of the above alkyl and alkoxy groups being optionally substituted by 1 to 3 groups OR⁸,

R⁴, R⁵, R⁶, R⁷ and R⁸ are independently hydrogen or C₁-C₆ alkyl, and wherein optionally one or more of the C7, C9, C11 and C13 double bonds is in cis-configuration,

for the cosmetic treatment or prophylaxis of wrinkles, skin aging and/or for thickening the epidermis.

The compounds of formula (I), wherein NR¹R² represents a residue 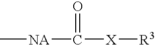
or at least one of R¹ and R² is a residue 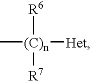
or wherein R1 and R2 form a ring structure, and wherein optionally one or more of the C7, C9, C11 and C13 double bonds is in cis-configuration, have not been used in the cosmetic field, and the present invention also provides the use of these compounds for providing a cosmetic effect.

The compounds of formula (I), wherein NR¹R² represents a residue 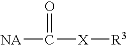
or at least one of R¹ and R² is a residue 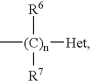
or wherein R¹ and R² form a ring structure, and wherein optionally one or more of the C7, C9, C11 and C13 double bonds is in cis-configuration, with the proviso that residue NR¹R² is not the residue of a single sulfur containing amino acid, are novel compounds, and the invention also provides these compounds and cosmetic compositions containing them.

As used in this specification, a C₁-C₃₀ hydrocarbon group is preferably a C₁-C₂₀-(more preferably C₁-C₆-)alkyl, C₂-C₂₀-(more preferably C₂-C₆-)alkenyl or C₂-C₂₀-(more preferably C₂-C₆-)alkynyl group, each of these groups can be straight chained or branched and can be substituted or unsubstituted. The substitution, if present, is preferably by a C₃-C₁₀-cycloalkyl group and/or a C₆-C₁₀-aryl group, and preferably 1, 2 or 3 substituents are present. It is also preferred that the above alkyl, alkenyl or alkynyl chains are interrupted by one or more C₃-C₁₀-cycloalkyl groups and/or C₆-C₁₀-aryl groups. The alkenyl groups comprise preferably not more than 5 double bonds, most preferably 1, 2 or 3 double bonds. The alkynyl groups comprise preferably not more than 5 triple bonds, more preferably 1, 2 or 3 triple bonds. Beside the triple bonds the alkynyl groups can also contain double bonds, and if such double bonds are present, preferably 1, 2 or 3 double bonds are present.

As used in this specification, a C₁-C₁₆-hydrocarbon group is preferably a C₁-C₁₆-alkyl, more preferably C₁-C₆-alkyl, a C₂-C₆-alkenyl or a C₂-C₆-alkynyl group, each of these groups can be straight chained or branched and can be substituted or unsubstituted. The substitution, if present, is preferably by a C₃-C₁₀-cycloalkyl group and/or a C₆-C₁₀-aryl group, and preferably 1, 2 or 3 substituents are present. It is also preferred that the above alkyl, alkenyl or alkynyl chains are interrupted by one or more C₃-C₁₀-cycloalkyl groups and/or C₆-C₁₀-aryl groups. The alkenyl groups comprise preferably not more than 3 double bonds, most preferably 1 or 2 double bonds. The alkynyl groups comprise preferably not more than 3 triple bonds, more preferably 1 or 2 triple bonds. Beside the triple bonds the alkynyl groups can also contain double bonds, and if such double bonds are present, preferably 1 or 2 double bonds are present.

In those embodiments in which residues R¹ and R² form together with the nitrogen atom to which they are attached a 5- to 8-membered ring, this ring is preferably 5- or 6-membered. The ring contains beside the nitrogen atom optionally 1 or 2 further heteroatoms, preferably 1 further heteroatom. The further heteroatom(s) is(are) nitrogen, oxygen or sulfur, preferably nitrogen or oxygen, most preferably oxygen. The ring is saturated or unsaturated, if the ring is unsaturated, it contains preferably 1 or 2 double bonds, more preferably 1 double bond, or it contains an aromatic unsaturation. Most preferably the ring is a morpholino ring. The ring can be unsubstituted or substituted, preferably the ring is unsubstituted. If the ring is substituted, the substituents are preferably bonded to carbon atoms of the ring structure and 1 to 3 substituents, more preferably 1 or 2 substituents, still more preferably 1 substituent, are present. The substituents are independently selected from C₁-C₆ alkyl groups, OR⁸ groups, or C₁-C₆ alkoxy groups, each of the above alkyl and alkoxy groups being optionally substituted by 1 to 3 groups OR⁸.

If one of residues R¹ and R² is a residue 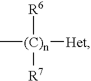
the other one is preferably hydrogen or a C₁-C₆ alkyl group, in particular hydrogen. Index n is 0 to 3, in particular 1 to 3, preferred 1 or 2, most preferred is n 1. Residues R⁶ and R⁷ are independently of each other hydrogen or C₁-C₆ alkyl. Most preferred is that not more than two of residues R⁶ and R⁷ are C₁-C₆ alkyl, and particularly preferred only one of residues R6 and R⁷ is C₁-C₆ alkyl. Most preferred, all residues R⁶ and R⁷ are hydrogen.

Residue Het is a ring structure with 5 to 8, in particular 5 or 6 ring atoms, which can be saturated or unsaturated. If the ring structure is unsaturated, it can contain I to 3, in particular 1 or 2, preferably 1 double bond or constitute an aromatic ring structure. The ring structure of Het contains 1 to 3 heteroatoms independently selected from nitrogen, oxygen and sulfur, particularly from nitrogen and oxygen, most preferably at least one heteroatom and preferably all heteroatoms are nitrogen. Particularly preferred are ring structures with 1 or 2 heteroatoms, more preferred are ring structures with 1 heteroatom. A particularly preferred ring structure is the pyridine or the pyrimidine structure. The ring structure Het can be substituted or unsubstituted, and if the structure is substituted, the substituents are preferably bonded to the carbon atoms of the ring structure. If the ring structure Het is substituted, it contains 1 to 4, preferably 2 to 4, most preferably 2 or 3 substituents. The substituents are independently selected from C₁-C₆ alkyl groups, OR⁸ groups, or C₁-C₆ alkoxy groups, each of the above alkyl and alkoxy groups being optionally substituted by 1 to 3 groups OR⁸. Most preferred substituent groups are hydroxyl groups and C₁-C₃ alkyl groups which are unsubstituted or substituted with a residue OR⁸. The alkyl and alkoxy groups are most preferably unsubstituted or substituted with 1 substituent.

Particularly preferred are those embodiments, wherein residue NR¹R² represents a residue NA—C(O)—X—R³, wherein X is O and R³ is hydrogen or a C₁-C₁₆-hydrocarbon, in particular a C₁-C₁₆— or a C₁-C₆-alkyl group. Those are compounds, wherein NR¹R² represents the residue of an amino acid or of a peptide which is bonded to the retinoyl moiety over the N-terminus of the amino acid or the peptide, and the peptide is composed of 2 to 6, that means, 2, 3, 4, 5 or 6 amino acids, and the C-terminus of the amino acid or the peptide is optionally esterified by a C₁-C₁₆-hydrocarbon group, particularly a C₁-C₆-alkyl group and wherein optionally one or more of C7, C9, C11 and C13 double bonds is in cis-configuration.

The residue PAG represents a polyalkylene glycol of the formula [symbol]
R^{a}—O [symbol] ₙ [symbol] R^{b}—O [symbol] ₘ,
wherein n and m are numbers of 0 to 100, with the proviso that n+m is a number of 1 to 150, preferably of 2 to 100. Residues R^{a} and R^{b} are independently C₁-C₆-alkyl residues, in particular C₂-C₄-alkyl residues which can be straight-chained or branched-chained. Particularly preferred is index m is 0 and index n is a number from 2 to 100. Most preferred residue PAG is a polyethylene glycol, a polypropylene glycol or a polytetramethylene glycol. Residue PAG is particularly preferred polyethylene glycol, which means that residue R^{a} is CH₂-CH₂, index n is a number from 2 to 100 and index m is 0.

It should be understood that the above definition of PAG comprises both, individual residues of PAG which are well defined by a certain individual number of n and m, but also residues of PAG in which the values of indices n and m are only statistical mean values and the residue PAG consists of a mixture of several molecules having different values for indices n and m. It is well known to a skilled person that due to the preparation of PAG residues those residues often constitute a statistical mixture with the above indices n and m only constituting statistical mean values.

If R³ is hydrogen or a C₁-C₁₆-hydrocarbon residue, then residue X is preferably oxygen.

Particularly preferred are those embodiments, wherein NR¹R² represents the residue of the amino acids Glycine, α- or β-Alanine, Valine, Leucine, Isoleucine, Proline, Phenylalanine, Tryptophan, Methionine, Selenomethionine, Serine, Threonine, Cysteine, Hydroxyproline, Asparagine, Glutamine, Aspartic acid, Glutamic acid, Lysine, Hydroxylysine, Histidine, Arginine, Ornithine, Citrulline, Taurine, Sarcosine and Statine, Norleucine, Norvaline, or 2-N-Methylnorleucine or of an ester thereof. Particular suitable are the natural isomers of the mentioned amino acids.

Preferred are furthermore those embodiments, wherein NR¹R² is a residue 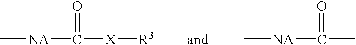
represents the residue of an amino acid as defined above.

More preferred are amino acids not containing a sulfur atom and most preferred are Phenylalanine, Glutamine and Hydroxyproline. Most preferred is Hydroxyproline.

Preferred are also embodiments, wherein NR¹R² represents the residue of a peptide selected from Carnosine (β-Ala-His), Homocarnosine, Balenine, Anserine, Aspartame (Phe-β-Ala), Arg-Pro or Pro-Arg, Gln-β-Ala-His, Glutathione (γ-Glu-Cys-Gly), Lys-Gly-His, Lys-Thr-Ser, Leu-Arg-Trp, Ile-Lys-Trp and Leu-Lys-Trp, Gly-Pro-Tyr, Lys-Pro-Val, Arg-Lys-Arg, Arg-Gly-Asp or Arg-Gly-Asp-Ser, Gly-Gln-Pro-Arg, Phe-Gly-Ala-Leu, PheGly-Gln-Pro-Arg, Arg-Pro-Phe-Phe, Tuftsine (Tyr-Lys-Pro-Arg), Regine (Gly-Gln-Pro-Arg), Phe-Tyr-Arg-Pro-Arg, Ala-Arg-Asp-Pro-Arg, Asn-Ser-Leu-Asp-Phe, Lys-Thr-Thr-Lys-Ser, Leu-Arg-Gly-Ile-Leu, Lys-Gly-lie-Leu, Lys-Leu-Asp-Ala-Pro-Thr or an ester thereof. Particular preferred are dipeptides.

Particularly preferred are those compounds of the present invention which have an octanol/water partition coefficient log POW in the range of 1 to 9, in particular of 2 to 8.5, preferably of 3 to 7. The log POW value can be measured experimentally by methods well known in the art or calculated (the clog POW value) by commercially available and well documented computer programs. The log POW values reported in this specification are clog POW values calculated by the computer program QikProp v2.1 (rel 8) of the company Schrödinger Software (New York and Portland, USA).

Preferred are furthermore those embodiments, wherein NR¹R² is a residue 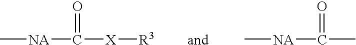
represents the residue of a peptide, in particular of a dipeptide, as defined above. Examples of the residue 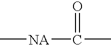
are e.g.

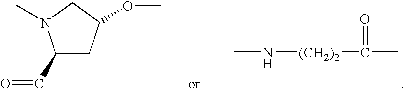

The compounds of the present invention, i.e. the conjugates of the amino acids or peptides with Retinoic Acid can be used alone or in mixtures. While the peptides which are useful for preparing the compounds of the present invention can be made using a direct synthesis method they can also be made by protein degradation. In case of using a protein hydrolysis process the resulting mixture can be used to make the conjugate and the resulting product would also be suitable according to this invention. However, the preferred embodiment is to use mixtures of not more than 3 compounds more preferred is the use of only one compound.

Some of the compounds of formula (I) are known compounds, and their preparation is described in the literature, e.g. in DE-A 40 32 187, in U.S. Pat. No. 4,190,594 and in DE-A 21 02 586, and the disclosure of these documents is included herein by reference. Those compounds of formula (I) which are novel compounds can be prepared by similar or other known methods or by methods which correspond to the methods exemplified in the experimental part of the present specification.

The compounds wherein residue R³ is a residue PAG-R⁴ can be prepared in analogy to the polyethoxylated retinamide derivatives disclosed in WO 99/50240, to which document it is particularly referred regarding the production process.

Instead of using tretinoin or tretinoin halide as in WO 99/50240, according to the present invention the condensation product of tretinoin and an amino acid or a peptide can be used, which condensation product can be prepared as exemplified in the present specification.

Particularly preferred compounds for use in the present invention (some of which are novel compounds) and the calculated clog POW values of these compounds (as far as available) are summarized in the following table:

| | clog | | |
|---|---|---|---|
| | POW | | |
| | (Qik | | |
| | Prop | | |
| | v2.1 | | |
| | (rel | Exam- | |
| Chemical Name | 8)) | ple | Structure |
| 2-{3-[3,7-Dimethyl-9- (2,6,6-trimethyl-cyclohex- 1-enyl)-nona-2,4,6,8- tetraenoylamino]- propionylamino}-3-(1H- imidazol-4-yl)-propionic acid | 5.55 | 6 | 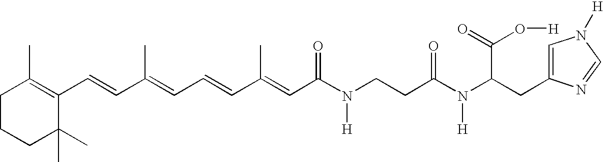 |
| 1-[3,7-Dimethyl-9-(2,6,6- trimethyl-cyclohex-1- enyl)-nona-2,4,6,8- tetraenoyl]-4-hydroxy- pyrrolidine-2-carboxylic acid | 4.686 | 2 | 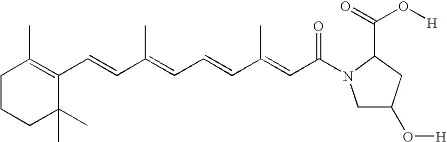 |
| 2-{3-[3,7-Dimethyl-9- (2,6,6-trimethyl-cyclohex- 1-enyl)-nona-2,4,6,8- tetraenoylamino]- propionylamino}-3-(1H- imidazol-4-yl)-propionic acid methyl ester | 6.504 | 5 | 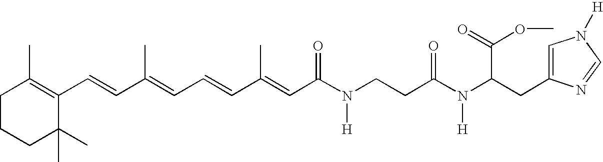 |
| 3,7-Dimethyl-9-(2,6,6- trimethyl-cyclohex-1- enyl)-nona-2,4,6,8- tetraenoic acid (3- hydroxy-5- hydroxymethyl-2-methyl- pyridin-4-ylmethyl)-amide | 5.986 | 7 | 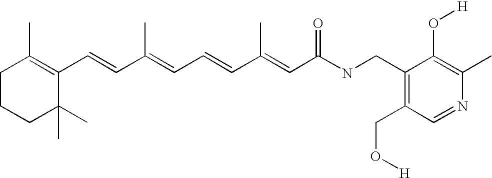 |
| 3-[3,7-Dimethyl-9-(2,6,6- trimethyl-cyclohex-1- enyl)-nona-2,4,6,8- tetraenoylamino]- propionic acid | 5.891 | 4 | 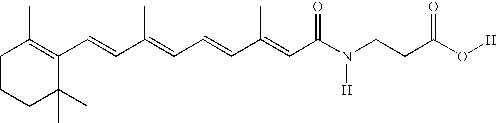 |
| 3,7-Dimethyl-1- morpholin-4-yl-9-(2,6,6- trimethyl-cyclohex-1- enyl)-nona-2,4,6,8- tetraen-1-one | 5.87 | | 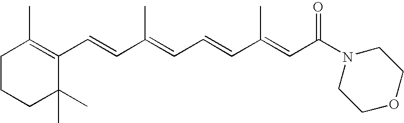 |
| 1-[3,7-Dimethyl-9-(2,6,6- trimethyl-cyclohex-1- enyl)-nona-2,4,6,8- tetraenoyl]-4-hydroxy- pyrrolidine-2-carboxylic acid ethyl ester | 6.083 | 1 | 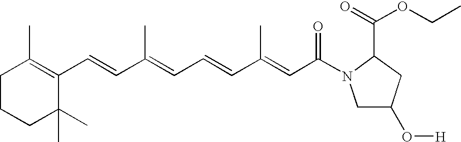 |
| 3,7-Dimethyl-9-(2,6,6- trimethyl-cyclohex-1- enyl)-nona-2,4,6,8- tetraenoic acid ethyl amide | 6.346 | | 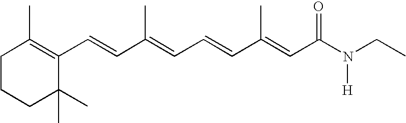 |
| 3-[3,7-Dimethyl-9-(2,6,6- trimethyl-cyclohex-1- enyl)-nona-2,4,6,8- tetraenoylamino]- propionic acid ethyl ester | 7.29 | 3 | 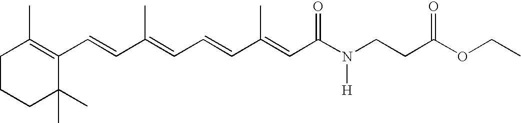 |
| 3,7-Dimethyl-9-(2,6,6- trimethyl-cyclohex-1- enyl)-nona-2,4,6,8- tetraenoic acid diethyl amide | 6.975 | | 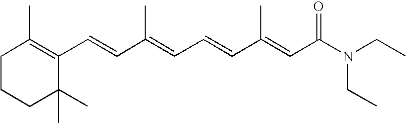 |
| 3,7-Dimethyl-9-(2,6,6- trimethyl-cyclohex-1- enyl)-nona-2,4,6,8- tetraenoic acid butyl amide | 7.157 | | 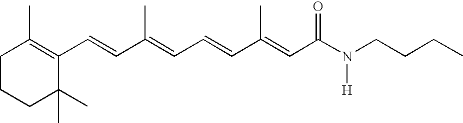 |
| Retinoyl- hydroxyprolin-O- PEG-2 | 6.421 | | 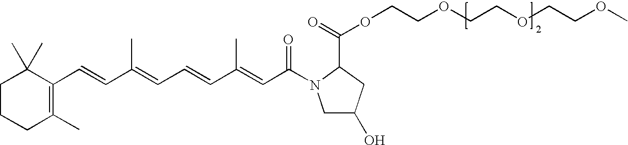 |
| Retinoyl- hydroxyprolin-O- PEG-10 | 7.944 | | 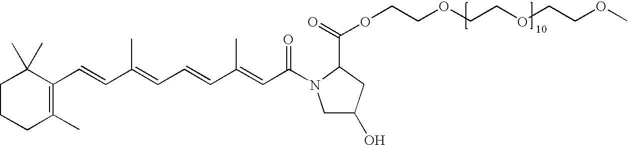 |
| Retinoyl- hydroxyprolin-O- PEG25 | n.d. | | 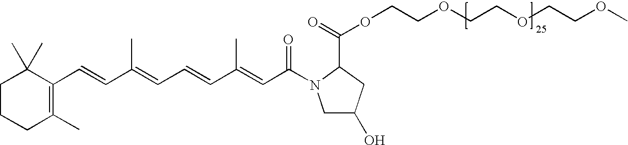 |
| Retinoyl- hydroxyprolin- NH-PEG-2 | 5.346 | | 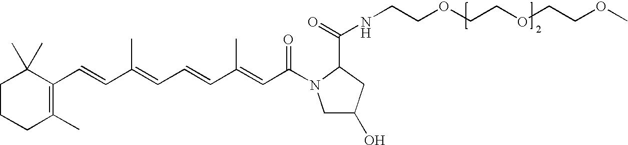 |
| Retinoyl- hydroxyprolin- NH-PEG-10 | 6.913 | | 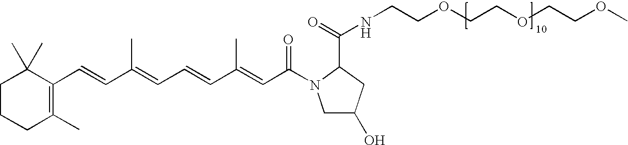 |
| Retinoyl- hydroxyprolin- NH-PEG-25 | n.d. | | 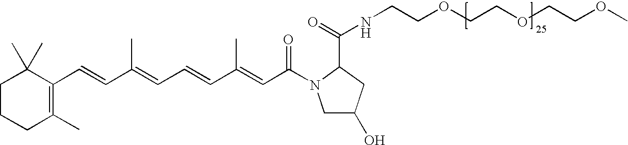 |

The present invention also provides compositions comprising at least one compound represented by general formula (I), and a cosmetically acceptable excipient or diluent.

The compounds of formula (I) are useful for providing a cosmetic effect, in particular for treatment or prophylaxis of wrinkles or dry skin or sensitive skin or any symptoms caused by negative developments of the physiological homeostasis of healthy skin, skin aging a thickening of the epidermis, anti-acne, the inhibition of senescence of skin cells, prevention or treatment of photo damage, prevention or treatment of oxidative stress phenomena, prevention or treatment of cellulite, prevention or treatment of pigmentation disorders and/or even the skin tone, prevention and treatment of disturbances in ceramide and lipid synthesis, prevention of excess sebum production, reduction of activities of matrix metallo proteases or other proteases in the skin, treatment and prevention of inflammatory skin conditions including atopic eczema, polymorphic light eruption, psoriasis, vertiligo, prevention and treatment of itchy or irritated skin, more preferably for the cosmetic treatment or prophylaxis of wrinkles, skin aging and/or for thickening the epidermis.

In case that the compounds of formula I bear one or more chiral centers the compounds represented by general formula (I) may be present in a racemic mixture, in a mixture of diastereomers or in excess of an enantiomer and/or a diastereomer. If one or more chiral centers are present the optical purity of the mixture is preferably ≧80% ee, more preferably ≧90% ee, most preferably ≧95% de. If two or more chiral centers are present the purity of the mixture is preferably ≧80% de, more preferably ≧90% de, most preferably ≧95% de.

The compositions of the present invention are cosmetic compositions or cosmetic preparations.

The term “cosmetic preparation” or “cosmetic composition” as used in the present application refers to cosmetic compositions as defined under the heading “Kosmetika” in Römpp Lexikon Chemie, 10th edition 1997, Georg Thieme Verlag Stuttgart, New York.

The compositions of the present invention contain the compound represented by general formula (I) with cosmetically acceptable excipients or diluents. If nothing else is stated, the excipients, additives, diluents, etc. mentioned in the following are suitable for cosmetic compositions.

If nothing else is stated, in this application parts and percentages are per weight and are based on the weight of the composition.

Preferably, the compositions of the present invention are topical compositions, such as liquid or solid oil-in-water emulsions, water-in-oil emulsions, multiple emulsions, microemulsions, PET-emulsions, bickering emulsions, hydrogels, alcoholic gels, lipogels, one or multiphase solutions, foams, ointments, plasters, suspensions, powders, cremes, cleanser, soaps and other usual compositions, which can also be applied by pens, as masks or as sprays.

The compositions of the invention can also contain usual cosmetic adjuvants and additives, such as preservatives/antioxidants, fatty substances/oils, water, organic solvents, silicones, thickeners, softeners, emulsifiers, sunscreens, cosmetic actives antifoaming agents, moisturizers, fragrances, surfactants, fillers, sequestering agents, anionic, cationic, nonionic or amphoteric polymers or mixtures thereof, propellants, acidifying or basifying agents, dyes, colorants, pigments or nanopigments, e.g. those suited for providing a photoprotective effect by physically blocking out ultraviolet radiation, or any other ingredients usually formulated into cosmetics.

The composition of the present invention can also contain additional pharmaceutically or cosmetically active ingredients, in particular for preventing or reducing acne, wrinkles, lines, atrophy inflammation, as well as topical anesthetics, artificial tanning agents and accelerators, antimicrobial agents, and antifungal agents, and sunscreening actives.

Examples are peptides (e.g., Matrixyl™ [pentapeptide derivative]), farnesol, bisabolol, phytanetriol, glycerol, urea, guanidine (e.g., amino guanidine); vitamins and derivatives thereof such as ascorbic acid, vitamin A (e.g., retinoid derivatives such as retinyl palmitate or retinyl propionate), vitamin E (e.g., tocopherol acetate), vitamin B₃ (e.g., niacinamide) and vitamin B₅ (e.g., octyl palmitate and tribehenin and sorbitan isostearate and palmitoyl-oligopeptide), anti-acne medicaments (resorcinol, salicylic acid, and the like); antioxidants (e.g., phytosterols, lipoic acid); flavonoids (e.g., isoflavones, phytoestrogens); skin soothing and healing agents such as aloe vera extract, allantoin and the like; chelators and sequestrants; and agents suitable for aesthetic purposes such as essential oils, fragrances, skin sensates, opacifiers, aromatic compounds (e.g., clove oil, menthol, camphor, eucalyptus oil, and eugenol), desquamatory actives, anti-acne actives, vitamin B₃ compounds, anti-oxidants, peptides, hydroxy acids, radical scavengers, chelators, farnesol, anti-inflammatory agents, topical anesthetics, tanning actives, skin lightening agents, anti-cellulite agents, flavonoids, antimicrobial actives, and antifungal actives, in particular bisabolol, alkyldiols such as 1,2-pentanediol, hexanediol or 1,2-octanediol, vitamins, panthenol, phytol, phytanetriol, ceramide and pseudoceramides, amino acids and bioactive peptides, protein hydrolysates, AHA acids, polyunsaturated fatty acids, plant extracts, DNA or RNA and their fragmentation products, carbohydrates.

Preferred additional active ingredients are also Biotin, lipoic acid, conjugated fatty acids, Carnitin, Acyl-Carnitin, Vit. E, Vit. A, Vit. C, B3, B6, B12, Panthenol, Kl, Phytantriol, Oligopeptides, Carnosin, Biochinonen, Phytofluen, Phytoen, folic acid and their corresponding derivatives.

The content of the active ingredients in the oral compositions of the present invention is usually about 1% to 90%, preferably about 10% to 80%, e.g. about 50% or more. The application is such that the desired effect occurs and depends on the patient and the desired effect. A usual daily dosage can be in a range from about 0.1 μg/day to 50 mg/day, e.g. about 20 μg/day to 2 mg/day.

Additionally the composition of the present invention may contain UV-A and UV-B filters. Examples of UV-B or broad spectrum screening agents, i.e. substances having absorption maximums between about 290 and 340 nm, which are preferred for combination with the compounds of the present invention, are the following organic and inorganic compounds:

Acrylates such as 2-ethylhexyl 2-cyano-3,3-diphenylacrylate (octocrylene, PARSOL® 340), ethyl 2-cyano-3,3-diphenylacrylate and the like;
Camphor derivatives such as 4-methyl benzylidene camphor (PARSOL® 5000), 3-benzylidene camphor, camphor benzalkonium methosulfate, polyacrylamidomethyl benzylidene camphor, sulfobenzylidene camphor, sulfomethyl benzylidene camphor, terephthalidene dicamphor sulfonic acid and the like;
Cinnamate derivatives such as octyl methoxycinnamate (PARSOL® MCX), ethoxyethyl methoxycinnamate, diethanolamine methoxycinnamate (PARSOL® Hydro), isoamyl methoxycinnamate and the like as well as cinnamic acid derivatives bond to siloxanes;
p-Aminobenzoic acid derivatives, such as p-aminobenzoic acid, 2-ethylhexyl p-dimethylaminobenzoate, N-oxypropylenated ethyl p-aminobenzoate, glyceryl p-aminobenzoate,
Benzophenones such as benzophenone-3, benzophenone-4, 2,2′, 4,4′-tetrahydroxy-benzophenone, 2,2′-dihydroxy-4,4′-dimethoxybenzophenone and the like;
Esters of Benzalmalonic acid such as di-(2-ethylhexyl) 4-methoxybenzalmalonate;
Esters of 2-(4-ethoxy-anilinomethylene)propanedioic acid such as 2-(4-ethoxy anilinomethylene)propanedioic acid diethyl ester as described in the European Patent Publication EP 0895 776;
Organosiloxane compounds containing benzmalonate groups as described in the European Patent Publications EP 0358584 B1, EP 0538431 B1 and EP 0709080 A1, in particular Parsol SLX;
Drometrizole trisiloxane (Mexoryl XL);
Pigments such as microparticulated TiO₂, and the like. The term “microparticulated” refers to a particle size from about 5 nm to about 200 nm, particularly from about 15 nm to about 100 nm. The TiO₂ particles may also be coated by metal oxides such as e.g. aluminium or zirconium oxides or by organic coatings such as e.g. polyols, methicone, aluminium stearate, alkyl silane. Such coatings are well known in the art.
Imidazole derivatives such as e.g. 2-phenyl benzimidazole sulfonic acid and its salts (PARSOL®HS). Salts of 2-phenyl benzimidazole sulfonic acid are e.g. alkali salts such as sodium- or potassium salts, ammonium salts, morpholine salts, salts of primary, sec. and tert. amines like monoethanolamine salts, diethanolamine salts and the like.
Salicylate derivatives such as isopropylbenzyl salicylate, benzyl salicylate, butyl salicylate, octyl salicylate (NEO HELIOPAN OS), isooctyl salicylate or homomenthyl salicylate (homosalate, HELIOPAN) and the like.
Triazine derivatives such as octyl triazone (UVINUL T-150), dioctyl butamido triazone (UVASORB HEB), bis ethoxyphenol methoxyphenyl triazine (Tinosorb S) and the like.
Encapsulated UV-filters such as encapsulated octyl methoxy cinnamate (Eusolex UV-pearls) and the like.

Examples of broad spectrum or UV A screening agents i.e. substances having absorption maximums between about 320 and 400 nm, which are preferred for combination with the compounds of the present invention are the following organic and inorganic compounds:
Dibenzoylmethane derivatives such as 4-tert. butyl-4′-methoxydibenzoyl-methane (PARSOL® 1789), dimethoxydibenzoylmethane, isopropyldibenzoylmethane and the like;
Benzotriazole derivatives such as 2,2′-methylene-bis-(6-(2H-benzotriazole-2-yl)-4-(1,1,3,3,-tetramethylbutyl)-phenol (TINOSORB M) and the like;
Phenylene-1,4-bis-benzimidazolsulfonic acids or salts such as 2,2-(1,4-phenylene)bis-(1H-benzimidazol-4,6-disulfonic acid) (Neoheliopan AP);
Amino substituted hydroxybenzophenones such as 2-(4-diethylamino-2-hydroxy-benzoyl)-benzoic acid hexylester as described in the European Patent Publication EP 1046391;
Pigments such as microparticulated ZnO or TiO₂ and the like. The term “microparticulated” refers to a particle size from about 5 nm to about 200 nm, particularly from about 15 nm to about 100 nm. The particles may also be coated by other metal oxides such as e.g. aluminium or zirconium oxides or by organic coatings such as e.g. polyols, methicone, aluminium stearate, alkyl silane. Such coatings are well known in the art.

As dibenzoylmethane derivatives have limited photostability it may be desirable to photostabilize these UV-A screening agents. Thus, the term “conventional UV-A screening agent” also refers to dibenzoylmethane derivatives such as e.g. PARSOL® 1789 stabilized by, e.g.,
3,3-Diphenylacrylate derivatives as described in the European Patent Publications EP-A 0 514 491 and EP-A 0 780 119;
Benzylidene camphor derivatives as described in the U.S. Pat. No. 5,605,680;
Organosiloxanes containing benzmalonate groups as described in the European Patent Publications EP-A 0358584, EP-A 0538431 and EP-A 0709080, in particular Parsol SLX.

A good overview of UV-A- and UV-B-filters which can be added to the compositions of the present invention can also be found in DE-A 103 27 432. All UV-filter compounds disclosed in this document are also useful as components for the compositions of the present invention and are included herein by reference.

The compositions of the present invention preferably contain one or more antioxidants/preservatives. Based on the invention all known antioxidants usually formulated into cosmetics can be used. Especially preferred are antioxidants chosen from the group consisting of amino acids (e.g. glycine, histidine, tyrosine, tryptophan) and their derivatives, imidazole (e.g. urocanic acid) and derivatives, peptides such as D,L-carnosine, D-carnosine, L-carnosine and derivatives (e.g. anserine), carotenoids, carotenes (e.g. (x-carotene, -carotene, lycopene) and derivatives, chlorogenic acid and derivatives, lipoic acid and derivatives (e.g. dihydrolipoic acid), aurothioglucose, propylthiouracil and other thiols (e.g. thioredoxine, glutathione, cysteine, cystine, cystamine and its glycosyl-, N-acetyl-, methyl-, ethyl-, propyl-, amyl-, butyl- and lauryl-, palmitoyl-; oleyl-, y-linoleyl-, cholesteryl- and glycerylester) and the salts thereof, dilaurylthiodipropionate, distearylthiodipropionate, thiodipropionic acid and its derivatives (ester, ether, peptides, lipids, nucleotides, nucleosides and salts) as well as sulfoximine compounds (such as buthioninsulfoximine, homocysteinsulfoximine, buthioninsulfone, penta-, hexa-, heptathioninsulfoximine) in very low compatible doses (e.g. pmol to μmol/kg), additionally (metal)-chelators (such as α-hydroxyfatty acids, palmic-, phytinic acid, lactoferrin), α-hydroxyacids (such as citric acid, lactic acid, malic acid), huminic acid, gallic acid, gallic extracts, bilirubin, biliverdin, EDTA, EGTA and its derivatives, unsaturated fatty acids and their derivatives (such as γ-linoleic acid, linolic acid, oleic acid), folic acid and its derivatives, ubiquinone and ubiquinol and their derivatives, vitamin C and derivatives (such as ascorbylpalmitate and ascorbyltetra-isopalmitate, Mg-ascorbylphosphate, Na-ascorbylphosphate, ascorbylacetate), tocopherol and derivates (such as vitamin-E-acetate), mixtures of nat. vitamin E, vitamin A and derivatives (vitamin-A-palmitate and -acetate) as well as coniferylbenzoate, rutinic acid and derivatives, α-glycosylrutin, ferulic acid, furfurylideneglucitol, carnosine, butylhydroxytoluene, butylhydroxyanisole, trihydroxybutyrophenone, urea and its derivatives, mannose and derivatives, zinc and derivatives (e.g. ZnO, ZnSO₄), selenium and derivatives (e.g. selenomethionine), stilbenes and derivatives (such as stilbeneoxide, trans-stilbeneoxide) and suitable derivatives (salts, esters, ethers, sugars, nucleotides, nucleosides, peptides and lipids) of the named active ingredients. One or more preservatives/antioxidants may be present in an amount about 0.01 wt. % to about 10 wt. % of the total weight of the composition of the present invention. Preferably, one or more preservatives/antioxidants are present in an amount about 0.1 wt. % to about 1 wt. %.

Typically topical formulations also contain surface active ingredients like emulsifiers, solubilizers and the like. An emulsifier enables two or more not miscible components to be combined homogeneously. Moreover, the emulsifier acts to stabilize the composition. Emulsifiers that may be used in the present invention in order to form O/W, W/O, O/W/O or W/O/W emulsions/microemulsions include sorbitan oleate, sorbitan sesquioleate, sorbitan isostearate, sorbitan trioleate, polyglyceryl-3-diisostearate, polyglycerol esters of oleic/isostearic acid, polyglyceryl-6 hexaricinolate, polyglyceryl-4-oleate, polyglyceryl-4 oleate/PEG-8 propylene glycol cocoate, oleamide DEA, TEA myristate, TEA stearate, magnesium stearate, sodium stearate, potassium laurate, potassium ricinoleate, sodium cocoate, sodium tallowate, potassium castorate, sodium oleate, and mixtures thereof. Further suitable emulsifiers are phosphate esters and the salts thereof such as cetyl phosphate (Amphisol® A), diethanolamine cetyl phosphate (Amphisol®), potassium cetyl phosphate (Amphisol® K), sodium glyceryl oleate phosphate, hydrogenated vegetable glycerides phosphate and mixtures thereof. Furthermore, one or more synthetic polymers may be used as an emulsifier. For example, PVP eicosene copolymer, acrylates/C₁₀₋₃₀ alkyl acrylate crosspolymer, acrylates/steareth-20 methacrylate copolymer, PEG-22/dodecyl glycol copolymer, PEG-45/dodecyl glycol copolymer, and mixtures thereof. The preferred emulsifiers are cetyl phosphate (Amphisol® A), diethanolamine cetyl phosphate (Amphisol®), potassium cetyl phosphate (Amphisol® K), PVP Eicosene copolymer, acrylates/C₁₀₋₃₀-alkyl acrylate crosspolymer, PEG-20 sorbitan isostearate, sorbitan isostearate, and mixtures thereof. The one or more emulsifiers are present in a total amount about 0.01 wt. % to about 20 wt. % of the total weight of the composition of the present invention. Preferably, about 0.1 wt. % to about 10 wt. % of emulsifiers are used.

The lipid phase of the topical compositions can advantageously be chosen from:
mineral oils and mineral waxes;
oils such as triglycerides of caprinic acid or caprylic acid, preferable castor oil;
oils or waxes and other natural or synthetic oils, in an preferred embodiment esters of fatty acids with alcohols e.g. isopropanol, propylene glycol, glycerin or esters of fatty alcohols with carboxylic acids or fatty acids;
alkyl benzoates; and/or
silicone oils such as dimethylpolysiloxane, diethylpolysiloxane, diphenylpolysiloxane, cyclomethicones
   and mixtures thereof.

Exemplary fatty substances which can be incorporated in the oil phase of the emulsion, micro-emulsion, oleo gel, hydrodispersion or lipodispersion of the present invention are advantageously chosen from esters of saturated and/or unsaturated, linear or branched alkyl carboxylic acids with 3 to 30 carbon atoms, and saturated and/or unsaturated, linear and/or branched alcohols with 3 to 30 carbon atoms as well as esters of aromatic carboxylic acids and of saturated and/or unsaturated, linear or branched alcohols of 3-30 carbon atoms. Such esters can advantageously be selected from octylpalmitate, octylcocoate, octylisostearate, octyldodecylmyristate, cetearylisononanoate, isopropyl-myristate, isopropylpalmitate, isopropylstearate, isopropyloleate, n-butylstearate, n-hexyllaureate, n-decyloleate, isooctylstearate, isononylstearate, isononylisononanoate, 2-ethyl hexylpalmitate, 2-ethylhexyllaurate, 2-hexyldecylstearate, 2-octyldodecylpalmitate, stearylheptanoate, oleyloleate, oleylerucate, erucyloleate, erucylerucate, tridecylstearate, tridecyltrimellitate, as well as synthetic, half-synthetic or natural mixtures of such esters e.g. jojoba oil.

Other fatty components suitable for use in the topical compositions of the present invention include polar oils such as lecithins and fatty acid triglycerides, namely triglycerol esters of saturated and/or unsaturated, straight or branched carboxylic acid with 8 to 24 carbon atoms, preferably of 12 to 18 carbon-atoms whereas the fatty acid triglycerides are preferably chosen from synthetic, half synthetic or natural oils (e.g. cocoglyceride, olive oil, sun flower oil, soybean oil, peanut oil, rape seed oil, sweet almond oil, palm oil, coconut oil, castor oil, hydrogenated castor oil, wheat oil, grape seed oil, macadamia nut oil and others); apolar oils such as linear and/or branched hydrocarbons and waxes e.g. mineral oils, vaseline (petrolatum); paraffins, squalane and squalene, polyolefins, hydrogenated polyisobutenes and isohexadecanes, favored polyolefins are polydecenes; dialkyl ethers such as dicaprylylether; linear or cyclic silicone oils such as preferably cyclomethicone (octamethylcyclotetrasiloxane; cetyldimethicone, hexamethylcyclotri-siloxane, polydimethylsiloxane, poly(methylphenylsiloxane) and mixtures thereof.

Other fatty components which can advantageously be incorporated in topical compositions of the present invention are isoeicosane; neopentylglycoldiheptanoate; propyleneglycol-dicaprylate/dicaprate; caprylic/capric/diglycerylsuccinate; butyleneglycol caprylate/caprate; C₁₂₋₁₃-alkyllactate; di-C₁₂₋₁₃-alkyltartrate; triisostearin; dipentaerythrityl hexacaprylat-/hexacaprate; propyleneglycolmonoisostearate; tricaprylin; dimethylisosorbid. Especially beneficial is the use of mixtures C₁₂₋₁₅-alkylbenzoate and 2-ethylhexylisostearate, mixtures C₁₂₋₁₅-alkylbenzoate and isotridecylisononanoate as well as mixtures of C₁₂₋₁₅-alkylbenzoate, 2-ethylhexylisostearate and isotridecylisononanoate.

The oily phase of the compositions of the present invention can also contain natural vegetable or animal waxes such as bee wax, china wax, bumblebee wax and other waxes of insects as well as shea butter and cocoa butter.

A moisturizing agent may be incorporated into a topical composition of the present invention to maintain hydration or rehydrate the skin. Moisturizers that prevent water from evaporating from the skin by providing a protective coating are called emollients. Additionally an emollient provides a softening or soothing effect on the skin surface and is generally considered safe for topical use. Preferred emollients include mineral oils, lanolin, petrolatum, capric/caprylic triglyceraldehydes, cholesterol, silicones such as dimethicone, cyclomethicone, almond oil, jojoba oil, avocado oil, castor oil, sesame oil, sunflower oil, coconut oil and grape seed oil, cocoa butter, olive oil aloe extracts, fatty acids such as oleic and stearic, fatty alcohols such as cetyl and hexadecyl (ENJAY), diisopropyl adipate, hydroxybenzoate esters, benzoic acid esters of C₉₋₁₅-alcohols, isononyl iso-nonanoate, ethers such as polyoxypropylene butyl ethers and polyoxypropylene cetyl ethers, and C₁₂₋₁₅-alkyl benzoates, and mixtures thereof. The most preferred emollients are hydroxybenzoate esters, aloe vera, C₁₂₋₁₅-alkyl benzoates, and mixtures thereof. An emollient is present in an amount of about 1 wt. % to about 20 wt. % of the total weight of the composition. The preferred amount of emollient is about 2 wt. % to about 15 wt. %, and most preferably about 4 wt. % to about 10 wt. %.

Moisturizers that bind water, thereby retaining it on the skin surface are called humectants. Suitable humectants can be incorporated into a topical composition of the present invention such as glycerin, polypropylene glycol, 1,2-pentandiol, polyethylene glycol, lactic acid, pyrrolidone carboxylic acid, urea, phospholipids, collagen, elastin, ceramides, lecithin sorbitol, PEG-4, and mixtures thereof. Additional suitable moisturizers are polymeric moisturizers of the family of water soluble and/or swellable/and/or with water gelating polysaccharides such as hyaluronic acid, chitosan and/or a fucose rich polysaccharide which is e.g. available as Fucogel®1000 (CAS-Nr. 178463-23-5) by SOLABIA S. One or more humectants are optionally present at about 0.5 wt. % to about 8 wt. % in a composition of the present invention, preferably about 1 wt. % to about 5 wt. %.

The aqueous phase of the preferred topical compositions of the present invention can contain the usual cosmetic or pharmaceutical additives such as alcohols, especially lower alcohols, preferably ethanol and/or isopropanol, low diols or polyols and their ethers, preferably propyleneglycol, glycerin, ethyleneglycol, ethyleneglycol monoethyl- or monobutylether, propyleneglycol monomethyl- or -monoethyl- or -monobutylether, diethyleneglycol monomethyl- or -monoethylether and analogue products, polymers, foam stabilizers; electrolytes and especially one or more thickeners. Thickeners that may be used in formulations of the present invention to assist in making the consistency of a product suitable include carbomer, silicium dioxide, magnesium and/or aluminium silicates, beeswax, stearic acid, stearyl alcohol polysaccharides and their derivatives such as xanthan gum, hydroxypropyl cellulose, polyacrylamides, acrylate crosspolymers preferably a carbomer, such as carbopole® of type 980, 981, 1382, 2984, 5984 alone or mixtures thereof. Suitable neutralizing agents which may be included in the composition of the present invention to neutralize components such as e.g. an emulsifier or a foam builder/stabilizer include but are not limited to alkali hydroxides such as a sodium and potassium hydroxide; organic bases such as diethanolamine (DEA), triethanolamine (TEA), aminomethyl propanol, and mixtures thereof; amino acids such as arginine and lysine and any combination of any foregoing. The neutralizing agent can be present in an amount of about 0.01 wt. % to about 8 wt. % in the composition of the present invention, preferably, 1 wt. % to about 5 wt. %.

The addition of electrolytes into the composition of the present invention may be necessary to change the behavior of a hydrophobic emulsifier. Thus, the emulsions/microemulsions of this invention may contain preferably electrolytes of one or several salts including anions such as chloride, sulfates, carbonate, borate and aluminate, without being limited thereto. Other suitable electrolytes can be on the basis of organic anions such as, but not limited to, lactate, acetate, benzoate, propionate, tartrate and citrate. As cations preferably ammonium, alkyl ammonium, alkali- or alkaline earth metals, magnesium-, iron- or zinc-ions are selected. Especially preferred salts are potassium and sodium chloride, magnesium sulfate, zinc sulfate and mixtures thereof. Electrolytes can be present in an amount of about 0.01 wt. % to about 8 wt. % in the composition of the present invention.

The topical compositions of the invention can preferably be provided in the form of a lotion, a thickened lotion, a gel, a cream, a milk, an ointment, a powder or a solid tube stick and can be optionally be packaged as an aerosol and can be provided in the form of a mousse, foam or a spray. The compositions according to the invention can also be in the form of a suspension or dispersion in solvents or fatty substances, or alternatively in the form of an emulsion or microemulsion (in particular of O/W or W/O type, O/W/O or W/O/W-type), such as a cream or a milk, a vesicular dispersion, in the form of an ointment, a gel, a solid tube stick or an aerosol mousse. The emulsions can also contain anionic, nonionic, cationic or amphoteric surfactants.

The topical application is preferably at least once per day, e.g. twice or triple times a day. Usually it takes at least two days until the desired effect is achieved. However, it can take several weeks or even months until the desired effect is achieved.

The amount of the topical composition which is to be applied to the skin depends on the concentration of the active ingredients in the compositions and the desired cosmetic or pharmaceutical effect. For example, application can be such that a crème is applied to the skin. A crème is usually applied in an amount of 2 mg crème/cm² skin. The amount of the composition which is applied to the skin is, however, not critical, and if with a certain amount of applied composition the desired effect cannot be achieved, a higher concentration of the active ingredients can be used e.g. by applying more of the composition or by applying compositions which contain more active ingredient.

According to the invention for preparing the compositions the active ingredients can be used as such or in an encapsulated form, for example in a liposomal form. Liposomes are preferably formed with lecithins with or without addition of sterols or phytosterols. The encapsulation of the active ingredients can be alone or together with other active ingredients.

In the composition of the invention, in particular the topical compositions of the invention, the compound of formula (I) is contained in an amount of preferably 0.001 wt.-% to about 10 wt.-%, based on the total weight of the composition. More preferably, the compound is contained in the composition in an amount of about 0.001 wt.-% to about 5 wt.-%, more preferably in an amount of about 00.1 wt.-% to about 0.5 wt.-% or 0.3 wt.-%, in particular in an amount of about 0.1 wt.-%, based on the total amount of the composition.

In case that the (preferably topical) composition of the invention contains a further active ingredient, this further active ingredient is contained in an amount of preferably 0.0001 wt.-% to about 50 wt.-%, based on the total weight of the composition. More preferably, the further active ingredient is contained in the composition in an amount of about 0.01 wt.-% to about 20 wt.-%, more preferably in an amount of about 0.01 wt.-% to about 1 wt.-%, in particular in an amount of about 0.1 wt.-%, based on the total amount of the composition.

Regarding the kind of the topical preparation and the preparation of the topical preparations as well as for further suitable additives, it can be referred to the pertinent literature, e.g. to Novak G. A., Die kosmetischen Präparate—Band 2, Die kosmetischen Präparate—Rezeptur, Rohstoffe, wissenschaftliche Grundlagen (Verlag fur Chem. Industrie H. Ziolkowski K G, Augsburg).

The compositions of the present invention can also be in the form of injectable compositions. The preparation of injectable compositions is known to a skilled person, and it can be referred to the pertinent literature, in particular to Remington already cited above.

The compounds of formula (I) can also be present as hydrates or solvates, and the hydrates and solvates of the active ingredients are also encompassed by the present invention. The amino acid conjugates can also be administered as metal salts, ammonium or guanidinum salts. Preferred metal cations are sodium, potassium, calcium or zinc.

The following examples exemplify the invention, but they should not be construed as limiting the invention.

### EXAMPLE 1

### Preparation of 1-[3,7-Dimethyl-9-(2,6,6-trimethyl-cyclohex-1-enyl)-nona-2,4,6,8-tetraenoyl]-4-hydroxy-pyrrolidine-2-carboxylic acid ethyl ester:

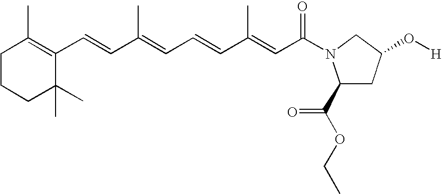 4-Hydroxy-pyrrolidine-2-carboxylic acid ethyl ester hydrochloride (651 mg, 3.33 mmol, 1.0 eq.) was dissolved in toluene (10 mL). The solution was cooled to 0° C. and NEt₃ (741 mg, 167.5 mmol, 2.2 eq.) was added, followed by retinoic acid chloride (1.06 g, 3.33 mmol, 1.0 eq.) in toluene (15 mL) over 10 min. The solution stirred 30 min at 0C and 30 min at room temperature. Water (50 mL) was then added, the layers separated and the aqueous layer was extracted three times with ethyl acetate (50 mL). The combined organic layers were washed once with an aqueous saturated NaCl solution and dried over Na₂SO₄. The solvent was evaporated under reduced pressure and the residue was purified by flash chromatography using TBME to yield the pure amide (500 mg, 34%) as a white solid. —R_{f} (TBME)=0.45; ¹H NMR (CDCl₃) δ=1.04 (s, 6H), 1.22 (t, J=7.1 Hz, 3H), 1.46-1.50 (m, 2H), 1.59-1.67 (m, 2H), 1.73 (s, 3H), 2.00 (s, 3H), 2.02-2.08 (m, 3H), 2.23-2.34 (m, 4H), 3.55-3.60 (m,1 H), 3.81-3.86 (m, 1H), 4.07-4.25 (q, J=7.1 Hz, 2H), 4.47-4.67 (m, 2H), 5.78-6.34 (m, 5H), 6.97 (dd, J=14.9, 11.3 Hz, 1H); IR (neat) cm⁻¹: v=3379, 2933, 1739, 1618, 1442, 1373, 1185, 1080, 968; MS (EI) m/z=441 (55) [M⁺], 29 (100) [C₂H₅⁺].

### EXAMPLE 2

### Preparation of 1-[3,7-Dimethyl-9-(2,6,6-trimethyl-cyclohex-1-enyl)-nona-2,4,6,8-tetraenoyl]-4-hydroxy-pyrrolidine-2-carboxylic acid:

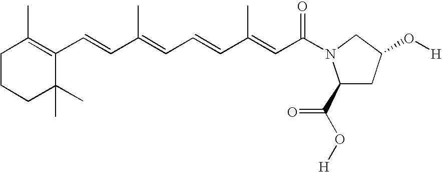 The ethyl ester obtained in example 1 (500 mg, 1.13 mmol, 1.0 eq.) was dissolved in EtOH (5 mL), NaOH (50 mg, 1.25 mmol, 1.1 eq.) in H₂0 (1 mL) was added and the solution stirred for 4 h at room temperature. The solvent was evaporated under reduced pressure, the residue was taken up in H₂O (10 mL) and CH₂Cl₂ (20 mL) was added. After acidification to pH=2 with 0.5 N H₂SO₄, the layers were separated and the aqueous layer was extracted twice with CH₂Cl₂ (10 mL). The organic layers were combined, washed once with Brine (10 mL) and dried over MgSO₄. The solvent was evaporated under reduced pressure to give the pure acid (250 mg, 54%) as a yellow solid. −¹H NMR (CDDl₃) δ=0.96 (s, 6H), 1.38-1.42 (m, 2H), 1.48-1.58 (m, 2H), 1.64 (s, 3H), 1.86-2.06 (m, 5H), 2.08-2.19 (m, 1H), 2.22 (s, 3H), 2.40-2.49 (m, 1H), 3.47-3.52 (m, 1H), 3.62-3.68 (m, 1H), 4.47-4.51 (m, 1H), 4.66-4.71 (m, 1H), 5.82 (s, 1H), 6.04-6.25 (m, 4H), 6.97 (dd, J=14.9, 11.4 Hz, 1H); ¹³C NMR (CDCl₃) δ=12.9, 14.4, 19.2, 21.7, 28.9 (2C), 33.1, 34.3, 36.1, 39.6, 55.3, 58.7, 69.6, 117.9, 128.9, 129.4, 130.1, 131.2, 134.9, 137.2, 137.7, 139.9, 151.7, 169.4, 172.6; IR (neat) cm⁻¹: v=3376, 2927, 2865, 1728, 1612, 1568, 1440, 1379, 1159, 1075, 964; MS (EI) m/z=412 (100) [M⁻-H].

### EXAMPLE 3

### Preparation of 3-[3,7-Dimethyl-9-(2,6,6-trimethyl-cyclohex-1-enyl)-nona-2,4,6,8-tetraenoylamino]-propionic acid ethyl ester:

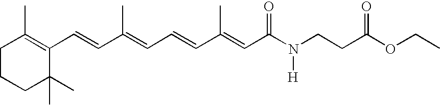 3-Amino-propionic acid ethyl ester hydrochloride (9.3 g, 60.5 mmol, 1.1 eq.) was dissolved in toluene (100 mL) and NEt₃ (12.2 g, 121.0 mmol, 2.2 eq.) in toluene (50 mL) was added with ice cooling. The solution stirred for 15 min at 15° C. and retinoic acid chloride (17.5 g, 55.0 mmol, 1.0 eq.) in toluene (220 mmol) was added slowly, keeping the temperature below 15° C. The solution stirred 1 h at 10° C., H₂O (300 mL) was then added and the solution was concentrated under reduced pressure. The solution was extracted twice with ethyl acetate (400 mL), the combined organic layer was dried over Na₂SO₄ and the solvent evaporated under reduced pressure. Purification by flash chromatography using hexane/ethyl acetate (7:3) yielded the pure amide (15.7 g, 71%) as a brown oil. −¹H NMR (CDCl₃) δ=1.04 (s, 6H), 1.24-1.30 (m, 3H), 1.44-1.50 (m, 2H), 1.59-1.67 (m, 2H), 1.72 (s, 3H), 2.00-2.06 (m, 5H), 2.36 (s, 3H), 2.58 (t, J=5.9 Hz, 2H), 3.58-3.61 (m, 2H), 4.09-4.21 (m, 2H), 5.66 (s, 1 H), 6.08-6.29 (m, 4H), 6.93 (dd, J=15.0, 11.3 Hz, 1H); MS (EI) m/z=339 (100) [M⁺].

### EXAMPLE 4

### Preparation of 3-[3,7-Dimethyl-9-(2,6,6-trimethyl-cyclohex-1-enyl)-nona-2,4,6,8-tetraenoylamino]-propionic acid

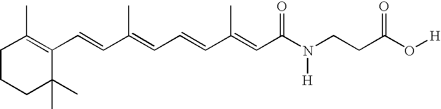 The ethyl ester of example 3 (39.9 g, 100.0 mmol, 1.0 eq.) was dissolved in EtOH (500 mL) and NaOH (4.0 g, 100.0 mmol, 1.0 eq.) in H₂O (100 mL) was added and the solution stirred over night at room temperature. The solution was concentrated under reduced pressure, H₂O (100 mL) and TBME (150 mL) were added. The layers were separated and ethyl acetate (400 mL) was added to the aqueous layer. After acidification to pH=1 with 0.5 N H₂SO₄, the layers were separated and the aqueous layer was extracted four times with ethyl acetate (350 mL). The organic layers were combined and the solvent was evaporated under reduced pressure and the residue was suspended in pentane (50 mL) and dried at high vacuum. The crude was recrystallized from ethyl acetate/acetonitrile/chloroform (400:150:100) to give the pure acid (18.3 g, 49%) as a yellow powder. −¹H NMR (CDCl₃) δ=0.95 (s, 6H), 1.38-1.42 (m, 2H), 1.51-1.59 (m, 2H), 1.64 (s, 3H), 1.92-1.98 (m, 5H), 2.28 (s, 3H), 2.58 (t, J=5.7 Hz, 2H), 3.49-3.55 (m, 2H), 5.58+5.72 (s, 1H), 6.00-6.27 (m, 5H), 6.81-7.01 (m, 1H); IR (neat) cm⁻¹: v=3309, 2931, 1708, 1549, 1183, 951; MS (EI) m/z=370 (100) [M⁻-H].

### EXAMPLE 5

### Preparation of 2-{3-[3,7-Dimethyl-9-(2,6,6-trimethyl-cyclohex-1-enyl)-nona-2,4,6,8-tetraenoylamino]-propionylamino}-3-(1H-imidazol-4-yl)-propionic acid methyl ester:

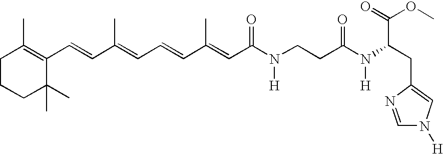 The acid of example 4 (12.3 g, 33.0 mmol, 1.0 eq) was dissolved in CH₂CI₂ (600 mL), EDC hydrochloride (7.6g, 39.6 mmol, 1.2 eq.), HOBt (6.2 g, 39.6 mmol, 1.2 eq.) and NEt3 (20.0 g, 19.8 mmol, 3.0 eq.) were added and the solution stirred for 1 h at room temperature. 2-Amino-3-(1 H-imidazol-4-yl)-propionic acid methyl ester hydrochloride (9.6 g, 39.6 mmol, 1.2 eq.) was added and the solution stirred at room temperature over night. The solvent was evaporated under reduced pressure, the residue was dissolved in ethyl acetate (2.0 L), the organic layer was washed twice with an aqueous saturated NaHCO₃ solution and dried over MgSO₄. After evaporation of the solvent under reduced pressure the residue was purified by flash chromatography using CH₂Cl₂/MeOH (9:1) yielding the pure product (6.5 g, 38%) as a pale yellow powder. —R_{f} (CH₂Cl₂/MeOH (9:1))=0.50; ¹H NMR (DMSO-d₆) δ=1.01 (s, 6H), 1.42-1.46 (m, 2H), 1.53-1.60 (m, 2H), 1.69 (s, 3H), 1.96-2.04 (m, 5H), 2.24-2.32 (m, 3H), 2.79-2.95 (m, 2H), 3.21-3.38 (m, 4H), 3.60 (s, 3H), 4.43-4.53 (m, 1H), 5.80+5.87 (s, 1H), 6.12-6.33 (m, 4H), 6.80 (s, 1H), 6.86-6.96 (m, 1H), 7.54 (s, 1H), 7.95-7.98 (m, 1H), 8.28-8.31 (m, 1H), 11.85 (br s, 1H); MS (ISP-MS) m/z=523 (100) [M⁺+H].

### EXAMPLE 6

### Preparation of 2-{3-[3,7-Dimethyl-9-(2,6,6-trimethyl-cyclohex-1-enyl)-nona-2,4,6,8-tetraenoylamino]-propionylamino}-3-(1H-imidazol-4-yl)-propionic acid

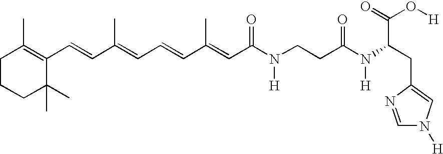 The methyl ester of example 5 (523 mg, 1.0 mmol, 1.0 eq.) was dissolved in MeOH (7 mL) and H₂0 (2 mL). An aqueous 0.5 M NaOH solution (2 mL, 1.0 mmol, 1.0 eq.) was added and the solution stirred over night at room temperature. Additional 1.0 M NaOH solution (0.2 mL, 0.2 mmol, 0.2 eq.) was added and the solution stirred again for 3 d at room temperature. The solvent was evaporated under reduced pressure and the residue dried at high vacuum yielding the sodium salt (530 mg, quant.) as a yellow powder. −¹H NMR (CD₃OD) δ=0.93 (s, 6H), 1.37-1.41 (m, 2H), 1.51-1.58 (m, 2H), 1.61 (s, 3H), 1.88 (s, 3H), 1.91-1.96 (m, 2H), 2.18 (s, 3H), 2.25-2.33 (m, 2H), 2.85-2.93 (m, 1H), 3.03-3.09 (m, 1H), 3.28-3.45 (m, 2H), 4.38-4.43 (m, 1H), 5.72 (s, 1H), 5.99-6.25 (m, 4H), 6.72 (s, IH), 6.87 (dd, J=15.0, 11.4 Hz, 1H), 7.41 (s, 1H); MS (EI) m/z=507 (100) [M⁻-H].

### EXAMPLE 7

### Preparation of 3,7-Dimethyl-9-(2,6,6-trimethyl-cyclohex-1-enyl)-nona-2,4,6,8-tetraenoic acid (3-hydroxy-5-hydroxymethyl-2-methyl-pyridin-4-ylmethyl)-amide

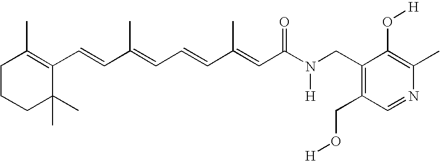 Pyridoxamine dihydrochloride (750 mg, 3.11 mmol, 1.0 eq.) was dissolved in CH₂Cl₂ (50 mL) and NEt₃ (1.9 g, 18.8 mmol, 6.0 eq.) was added and the solution stirred for 15 min at room temperature. In a second flask retinoic acid was dissolved in CH₂Cl₂ (200 mL) and activated with EDC hydrochloride (715 mg, 3.73 mmol, 1.2 eq.) and HOBt (586 mg, 3.73 mmol, 1.2 eq.). The solution stirred for 10 min at room temperature before the pyridoxamine solution was added over 10 min and the resulting solution stirred over night at room temperature. The solvent was evaporated under low pressure, the residue was taken up in ethyl acetate (400 mL), the organic layer was washed twice with 100 mL of a 10% aqueous NaHCO₃ solution and dried over Na₂SO₄. The solvent was evaporated under low pressure and the residue was purified by flash chromatography using ethyl acetate to yield the pure amide (630 mg, 45%) as a pale yellow solid. −R_{f} (ethyl acetate)=0.23; ¹H NMR (DMSO-d₆) δ=1.01 (s, 6H), 1.41-1.45 (m, 2H), 1.52-1.59 (m, 2H), 1.68 (s, 3H), 1.97 (s, 3H), 1.99-2.02 (m, 2H), 2.31 (s, 3H), 2.34 (s, 3H), 4.32-4.37 (m, 2H), 4.57 (d, J=5.4 Hz, 2H), 5.21 (t, J=5.4 Hz, 1H), 5.88 (s, 1H), 6.12-6.33 (m, 4H), 6.97 (dd, J=15.0, 11.4 Hz, 1H), 7.90 (s, 1H), 8.90-8.94 (m, 1H), 10.32 (s, 1H); MS (EI) m/z=450 (100) [M⁺].

### EXAMPLE 8

### Anti-Aging Cream

O/W Emulsion with Retinoyl-Hydroxyprolin-Ethylester (Example 1)

| Ingredients | % (w/w) |
|---|---|
| Glyceryl Myristate | 4.00 |
| Cetyl Alcohol | 2.00 |
| Steareth-2 | 2.00 |
| Steareth-21 | 2.00 |
| Isopropyl Myristate | 5.00 |
| Caprylic/Capric Triglyceride | 8.00 |
| BHT | 0.05 |
| Dimethicone | 2.00 |
| Phenoxyethanol & Methylparaben & Ethylparaben & | 0.80 |
| Butylparaben & Propylparaben & Isobutylparaben | |
| Retinoyl-Hydroxyprolin-Ethylester (Example 1) | 0.10 |
| Water | Ad. 100 |
| Xanthan Gum | 0.50 |
| Disodium EDETA | 0.10 |
| Propylene Glycol | 4.00 |

### EXAMPLE 9

### Around the Eye Reshaping Contour Gel

Gel with Retinoyl-Carnosin-Methylester (Example 5)

| Ingredients | % (w/w) |
|---|---|
| Water | Ad. 100 |
| Butylene Glycol | 4.00 |
| Acrylates/C10-30 Alkyl Acrylate Crosspolymer | 0.60 |
| NaOH 30% | 0.40 |
| Cyclomethicone | 5.00 |
| Disodium EDTA | 0.10 |
| Sodium Ascorbyl Phosphate | 0.20 |
| D-Panthenol | 0.50 |
| Phenoxyethanol & Methylparaben & Ethylparaben & | 0.80 |
| Propylparaben & Butylparaben & Isopropylparaben | |
| Glycerin | 3.00 |
| Polysorbate 20 | 0.80 |
| Retinoyl-Carnosin-Methylester (Example 5) | 0.10 |
| Tocopheryl Acetate | 0.10 |

### EXAMPLE 10

### Anti-Aging Facial Moisturizer

O/W Emulsion with Retinoic Acid N-ethyl Amide

| Ingredients | % (w/w) |
|---|---|
| Glyceryl Myristate | 5.00 |
| Cetyl Alcohol | 2.00 |
| Cetyl Phosphate | 2.00 |
| Isopropyl Myristate | 8.00 |
| Polysilicone-15 | 4.00 |
| Ethylhexyl Methoxycinnamate | 4.00 |
| Butyl Methoxydibenzoylmethane | 1.00 |
| Tocopheryl Acetate | 0.30 |
| Almond Oil | 1.00 |
| BHT | 0.05 |
| Phenoxyethanol & Methylparaben & Ethylparaben & | 0.60 |
| Propylparaben & Butylparaben & Isopropylparaben | |
| Tromethamine | 0.90 |
| Water | Ad. 100 |
| L-Carnosine | 0.20 |
| D-Panthenol | 1.00 |
| Disodium EDTA | 0.10 |
| Propylene Glycol | 4.00 |
| Polyacrylamide & C13-14 Isoparaffin & Laureth-7 | 2.00 |
| Retinoic Acid N-ethyl amide | 0.10 |
| Triethanolamine | q.s. |

### EXAMPLE 11

### Night Repair Cream

W/O Emulsion with Retinoyl-Hydroxyprolin-Ethylester (Example 1)

| Ingredients | % (w/w) |
|---|---|
| Polyglyceryl-2 Dipolyhydroxystearate | 4.00 |
| Polyglyceryl-3 Diisostearate | 2.00 |
| Beeswax | 2.00 |
| Zinc Stearate | 2.00 |
| Caprylic/Capric Triglyceride | 3.00 |
| Cetearyl Isononanoate | 8.00 |
| Dicaprylyl Ether | 5.00 |
| BHT | 0.05 |
| Phenoxyethanol & Methylparaben & Ethylparaben & | 0.60 |
| Propylparaben & Butylparaben & Isopropylparaben | |
| Water | Ad. 100 |
| D-Panthenol | 0.20 |
| Disodium EDTA | 0.10 |
| Propylene Glycol | 4.00 |
| Retinoyl-Hydroxyprolin-Ethylester (Example 1) | 0.10 |

Wrinkle Reduction Assay

The ability of the compounds and compositions of the present invention to reduce skin wrinkles can be assessed by profilometric methods described in “Skin topography measurement by interference fringe projection: a technical validation”. (Lagarde J M; Rouvrais C; Black D; Diridollou S; Gall Y, Skin research and technology: official journal of International Society for Bioengineering and the Skin (ISBS) [and] International Society for Digital Imaging of Skin (ISDIS) [and] International Society for Skin Imaging (ISSI) (2001 May), 7(2), 112-21 or “Direct and non-direct measurement techniques for analysis of skin surface topography”. Fischer T W; Wigger-Alberti W; Elsner P., Skin pharmacology and applied skin physiology (1999 January-April), 12(1-2), 1-11.

1. A method for the cosmetic treatment or prophylaxis of wrinkles, skin aging and/or thickening of the epidermis comprising applying to the skin an effective amount of a compound represented by general formula (I) 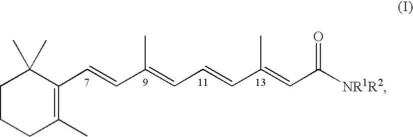
wherein
R¹ and R² independently of each other represent hydrogen, or a C₁-C₃₀-hydrocarbon group or a residue 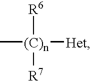
or R¹ and R² form together with the nitrogen atom to which they are attached a 5- to 8-membered saturated or unsaturated ring which contains besides the nitrogen atom carbon atoms and optionally 1 or 2 further heteroatoms selected from nitrogen, oxygen and sulfur atoms and which is unsubstituted or substituted with 1 to 3 substituents, independently selected from C₁-C₆ alkyl groups, OR⁸ groups, or C₁-C₆ alkoxy groups, each of the above alkyl and alkoxy groups being optionally substituted by 1 to 3 groups OR⁸, or
NR¹R² represents a residue 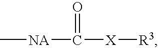
wherein 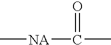
represents the residue of an amino acid or of a peptide which is bonded to the moiety 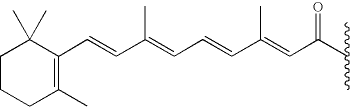
over the N-terminus of the amino acid or the peptide and the peptide is composed of 2 to 6, that means 2, 3, 4, 5 or 6 amino acids,
—X— is—O— or —NR⁵—
R³ is hydrogen, a C₁-C₆ hydrocarbon residue or a residue PAG-R⁴,
PAG is a residue of a polyalkylene glycol,
n is an integer of 0 to 3,
Het is a 5 to 8-membered saturated or unsaturated heterocycle which contains 1 to 3 heteroatoms, independently selected from nitrogen, oxygen and sulfur and which is optionally substituted with 1 to 4 substituents, independently selected from C₁-C₆ alkyl groups, OR⁸ groups, or C₁-C₆ alkoxy groups, each of the above alkyl and alkoxy groups being optionally substituted by 1 to 3 groups OR⁸,
R⁴, R⁵, R⁶, R⁷ and R⁸ are independently hydrogen or C₁-C₆ alkyl,
and wherein optionally one or more of the C7, C9, C11 and C13 double bonds is in cis-configuration.

2. Method according to claim 1, wherein R¹ and R² independently of each other represent hydrogen, a branched or straight chain C₁-C₂₀ alkyl group, a branched or straight chain C₂-C₂₀-alkenyl group or a branched or straight chain C₂-C₂₀-alkynyl group, wherein the alkenyl group has 1 to 5 double bonds and the alkynyl group has 1 to 5 triple bonds and wherein each of the above alkyl, alkenyl and alkynyl groups is optionally substituted by a C₃-C₁₀-cycloalkyl group or a C₆-C₁₀-aryl group and wherein optionally one or more of the C7, C9, C11 and C13 double bonds is in cis-configuration.

3. Method according to claim 1, wherein residue R¹ is H and residue R² is different from H.

4. Method according to claim 1, wherein NR¹R² represents the residue of an amino acid or of a peptide which is bonded over the N-terminus of the amino acid or the peptide and the peptide is composed of 2 to 6, that means 2, 3, 4, 5 or 6 amino acids and the C-terminus of the amino acid or the peptide is optionally esterified by a C₁-C₁₆ hydrocarbon group.

5. Method according to claim 4, wherein the C-terminus of the amino acid or the peptide is esterified with a C₁-C₁₆ alkyl residue.

6. Method according to claim 4, wherein the amino acid is selected from Glycine, α- or β-Alanine, Valine, Leucine, Isoleucine, Proline, Phenylalanine, Tryptophan, Methionine, Selenomethionine, Serine, Threonine, Cysteine, Hydroxyproline, Asparagine, Glutamine, Aspartic acid, Glutamic acid, Lysine, Hydroxylysine, Histidine, Arginine, Ornithine, Citrulline, Taurine, Sarcosine and Statine, Norleucine, Norvaline, or 2-N-Methylnorleucine.

7. Method according to claim 6 where NR¹R² represents Hydroxyproline or an ester of Hydroxyproline.

8. Method according to claim 4, wherein —NR¹R² represents the residue of a dipeptide which is optionally esterified by a C₁-C₁₆-hydrocarbon group.

9. Method according to claim 1, wherein residue NR¹R² represents a residue —NA—C(O)—X—R³, wherein —NA—C(O)— represents the residue of an amino acid or of a peptide which is bonded to the retinoyl moiety over the N-terminus of the amino acid or the peptide, and the peptide is composed of 2 to 6, that means 2, 3, 4, 5 or 6 amino acids, X is O or NR⁵, and R³ is a residue PAG-R⁴, wherein PAG is a residue of a polyalkylene glycol and R⁴ is hydrogen or C₁-C₆-alkyl.

10. Method according to claim 9, wherein PAG is a residue of formula [symbol]
R^{a}—O [symbol] ₙ [symbol] R^{b}—O [symbol] ₘ,
wherein residue R^{a} and R^{b} independently are branched or straight-chain C₁-C₆-alkyl residues, n and m are numbers of 0 to 100, and n+m is 1 to 150.

11. Method according to claim 10, wherein PAG is a polyethylene glycol residue having 2 to 100 ethylene glycol units.

12. A method for the cosmetic treatment or Prophylaxis of wrinkles, skin aging and/or thickening of the epidermis comprising applying to the skin a cosmetically effective amount of a compound represented by general formula (I) 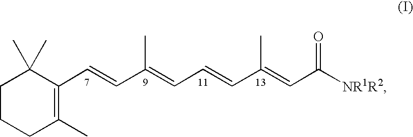
wherein
R¹ represents hydrogen, or a C₁-C₃₀-hydrocarbon group or a residue 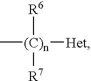
and R² represents a residue 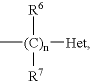
or R¹ and R² form together with the nitrogen atom to which they are attached a 5- to 8-membered saturated or unsaturated ring which contains besides the nitrogen atom carbon atoms and optionally 1 or 2 further heteroatoms selected from nitrogen, oxygen and sulfur atoms and which is unsubstituted or substituted with 1 to 3 substituents, independently selected from C₁-C₆ alkyl groups, OR⁸ groups, or C₁-C₆ alkoxy groups, each of the above alkyl and alkoxy groups being optionally substituted by 1 to 3 groups OR⁸, or
NR¹R² represents a residue 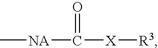
wherein 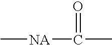
represents the residue of an amino acid or of a peptide which is bonded to the moiety 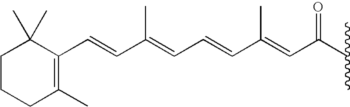
over the N-terminus of the amino acid or the peptide and the peptide is composed of 2 to 6, that means 2, 3, 4, 5 or 6 amino acids,
—X— is —O— or —NR⁵—
R³ is hydrogen, a C₁-C₁₆ hydrocarbon residue or a residue PAG-R⁴,
PAG is a residue of a polyalkylene glycol,
n is an integer of o to 3,
Het is a 5 to 8-membered saturated or unsaturated heterocycle which contains 1 to 3 heteroatoms, independently selected from nitrogen, oxygen and sulfur and which is optionally substituted with 1 to 4 substituents, independently selected from C₁-C₆ alkyl groups, OR⁸ groups, or C₁-C₆ alkoxy groups, each of the above alkyl and alkoxy groups being optionally substituted by 1 to 3 groups OR⁸,
R⁴, R⁵, R⁶, R⁷ and R⁸ are independently hydrogen or C₁-C₆ alkyl,
and wherein optionally one or more of the C7, C9, C11 and C13 double bonds is in cis-configuration.

13. Method according to claim 12, wherein the cosmetic effect is treatment or prophylaxis of wrinkles or dry skin or sensitive skin or any symptoms caused by negative developments of the physiological homeostasis of healthy skin, skin aging, a thickening of the epidermis, anti-acne, the inhibition of senescence of skin cells, prevention or treatment of photodamage, prevention or treatment of oxidative stress phenomena, prevention or treatment of cellulite, prevention or treatment of pigmentation disorders and/or even the skin tone, prevention and treatment of disturbances in ceramide and lipid synthesis, prevention of excess sebum production, reduction of activities of matrix metallo proteases or other proteases in the skin, treatment and prevention of inflammatory skin conditions including atopic eczema, polymorphic light eruption, psoriasis, vertiligo, prevention and treatment of itchy or irritated skin.

14. Method according to claim 12, wherein the C-terminus of the amino acid or the peptide is esterified with a C₁-C₁₆ alkyl residue.

15. Method We according to claim 12, characterized in wherein —NR¹R² represents the residue of an amino acid selected from Glycine, α- or β-Alanine, Valine, Leucine, Isoleucine, Proline, Phenylalanine, Tryptophan, Methionine, Selenomethionine, Serine, Threonine, Cysteine, Hydroxyproline, Asparagine, Glutamine, Aspartic acid, Glutamic acid, Lysine, Hydroxylysine, Histidine, Arginine, Ornithine, Citrulline, Taurine, Sarcosine and Statine, Norleucine, Norvaline, or 2-N-Methylnorleucine, which is optionally esterified by a C₁-C₁₆-hydrocarbon group.

16. Method according to claim 15 where NR¹R² represents Hydroxyproline or an ester of Hydroxyproline.

17. Method according to claim 12, characterized in wherein —NR¹R² represents the residue of a dipeptide which is optionally esterified by a C₁-C₁₆-hydrocarbon group.

18. Method according to claim 12, wherein residue NR¹R² represents a residue —NA—C(O)—X—R³, wherein —NA—C(O)— represents the residue of an amino acid or of a peptide which is bonded to the retinoyl moiety over the N-terminus of the amino acid or the peptide, and the peptide is composed of 2 to 6, that means 2, 3, 4, 5 or 6 amino acids, X is O or NR⁵, and R³ is a residue PAG-R⁴, wherein PAG is a residue of a polyalkylene glycol and R⁴ is hydrogen or C₁-C₆-alkyl.

19. Method according to claim 18, wherein PAG is a residue of formula [symbol]
R^{a}—O [symbol] ₙ [symbol] R^{b}—O [symbol] ₘ,
wherein residue R^{a} and R^{b} independently are branched or straight-chain C₁-C₆-alkyl residues, n and m are numbers of 0 to 100, and n +m is 1 to 150.

20. Method according to claim 19, wherein PAG is a polyethylene glycol residue having 2 to 100 ethylene glycol units.

21. Method according to claim 12, wherein residue R² is a residue 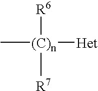
and residue R¹ is hydrogen or a C₁-C₆ alkyl group.

22. Method according to claim 21, wherein index n is 1 or2.

23. Method according to claim 21, wherein not more than one of residues R⁶ and R⁷ is different from hydrogen.

24. Method according to claim 21, wherein residue Het has 5 or 6 ring atoms.

25. Method according to claim 24, wherein residue Het is an optionally substituted aromatic heterocycle.

26. Method according to claim 21, wherein residue Het is a heterocycle which is substituted by 2 or 3 substituents.

27. Compound represented by general formula (I) 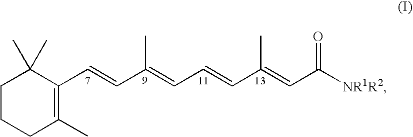
wherein NR¹R² represents a residue 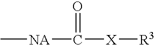
wherein 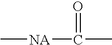
represents the residue of an amino acid or of a peptide which is bonded to the moietv 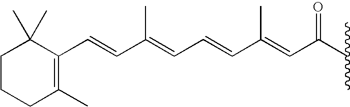
over the N-terminus of the amino acid or the peptide and the peptide is composed of 2 to 6, that means 2, 3. 4, 5 or 6 amino acids,
—X— is —O— or —NR⁵—
R³ is hydrogen, a C₁-C₁₆ hydrocarbon residue or a residue PAG-R⁴,
PAG is a residue of a polyalkylene glycol,
n is an integer of 0 to 3,
Het is a 5 to 8-membered saturated or unsaturated heterocycle which contains 1 to 3 heteroatoms, independently selected from nitrogen, oxygen and sulfur and which is optionally substituted with 1 to 4 substituents, independently selected from C₁-C₆ alkyl groups, OR⁸ groups, or C₁-C₆ alkoxy groups, each of the above alkyl and alkoxy groups being optionally substituted by 1 to 3 groups OR⁸,
R⁴, R⁵, R⁶, R⁷ and R⁸ are independently hydrogen or C₁-C₆ alkyl, and wherein
optionally one or more of the C7, C9, C11 and C13 double bonds is in cis-configuration, with the proviso that residue NR¹R² is not the residue of a single sulfur containing amino acid.

28. Compound according to claim 27, wherein the C-terminus of the amino acid or the peptide is esterified with a C₁-C₁₆ hydrocarbon group.

29. Compound according to claim 27, wherein —NR¹R² represents the residue of an amino acid selected from Glycine, α- or β-Alanine, Valine, Leucine, Isoleucine, Proline, Phenylalanine, Tryptophan, Serine, Threonine, Hydroxyproline, Asparagine, Glutamine, Aspartic acid, Glutamic acid, Lysine, Hydroxylysine, Histidine, Arginine, Ornithine, Citrulline, Taurine, Sarcosine and Statine, Norleucine, Norvaline, or 2-N-Methylnorleucine which is optionally esterified by a C₁-C₁₆-hydrocarbon group.

30. Compound according to claim 29 where —NR¹R² represents Hydroxyproline or an ester of Hydroxyproline.

31. Compound according to claim 27, characterized in that wherein —NR¹R² represents the residue of a dipeptide which is optionally esterified by a C₁-C₁₆-hydrocarbon group.

32. Compound according to claim 27, wherein residue NR¹R² represents a residue —NA—C(O)—X—R³, wherein —NA—C(O)— represents the residue of an amino acid or of a peptide which is bonded to the retinoyl moiety over the N-terminus of the amino acid or the peptide, and the peptide is composed of 2 to 6, that means 2, 3, 4, 5 or 6 amino acids, X is O or NR⁵, and R³ is a residue PAG-R⁴, wherein PAG is a residue of a polyalkylene glycol and R⁴ is hydrogen or C₁-C₆-alkyl.

33. Compound according to claim 32, wherein PAG is a residue of formula [symbol]
R^{a}—O [symbol] ₙ [symbol] R^{b}—O [symbol] ₘ,
wherein residues R^{a} and R^{b} independently are branched or straight-chain C₁-C₆-alkyl residues, n and m are numbers of 0 to 100, and n+m is 1 to 150.

34. Compound according to claim 33, wherein PAG is a polyethylene glycol residue having 2 to 100 ethylene glycol units.

35. Compound according to claim 27, wherein residue R² is a residue 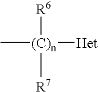
and residue R¹ is hydrogen or a C₁-C₆ alkyl group.

36. Cosmetic composition comprising at least one compound according to claim 27 and a cosmetically acceptable excipient or diluent.

37. Composition according to claim 36, wherein the composition is a topical composition.

38. Cosmetic composition according to claim 36, wherein the composition contains the compound of formula (I) in a concentration of 0.001 to 10 wt.-%, based on the weight of the composition.

39. Cosmetic composition according to claim 38, wherein the compound of formula (I) is present in a concentration of 0.01 to 0.5 wt.-%, based on the weight of the composition.
